(19) United States
(12) Patent Application Publication
(10) Pub. No.: US 2008/0125837 A1
(43) Pub. Date: May 29, 2008

(54) Noninvasive method for site-specific fat reduction with catalyst

(75) Inventors: Steven C. Shanks, McKinney, TX (US); Rodrigo Neira, Red Deer (CA)

Correspondence Address: ETHERTON LAW GROUP, LLC, 5555 E. VAN BUREN STREET, SUITE 100, PHOENIX, AZ, 85008, US

(73) Assignee: Therapy Products, Inc.

(21) Appl. No.: 12/012,147

(22) Filed: Jan. 31, 2008

Related U.S. Application Data
(63) Continuation-in-part of application No. 11/053,369, filed on Feb. 7, 2005.
(60) Provisional application No. 60/542,720, filed on Feb. 6, 2004.

(51) Int. Cl. A61N 5/067

(52) U.S. Cl. 607/89

(57) ABSTRACT

A noninvasive method of reducing fat from targeted regions of a patient's body by applying low-level laser energy externally through the skin of the patient to the targeted areas and treating the patient with a catalyst to prevent the lasered fat cells from functioning normally. Sufficient laser energy is applied to release at least a portion of intracellular fat into the interstitial space and the preferred embodiment uses laser light at about 635 nm. The catalyst is any combination of sterile water and saline solution that kills the lasered fat cells or prevents recuperation of the lasered cells before they are removed from the body through the body's natural functions. Preferably the catalyst is a hypotonic solution.

### CROSS-REFERENCE TO RELATED APPLICATIONS

This application is a continuation-in-part-of co-pending U.S. application Ser. No. 11/053,369 filed Feb. 7, 2005, which claims the benefit of U.S. Provisional Application No. 60/542,720 filed Feb. 6, 2004.

### FIELD OF INVENTION

This invention relates to a method for non-invasive, non-traumatic shaping and contouring of a human body by external means. In particular, this invention relates to the application of laser energy to targeted external regions of a patient's body to reduce fat, aided by a catalyst.

### BACKGROUND

There is a great demand to be slimmer. Many people resort to the cosmetic surgical procedure known as liposuction, wherein excess adipose tissue, also known as fat, is suctioned from the body of a patient. The typical purpose of the liposuction procedure is to leave the patient thinner, with aesthetically more appealing body contours. For example, liposuction is often performed on patients to remove excess fat in the abdominal, buttock, thigh, breast and arm regions of the body.

Adipose tissue is made of adipocytes, or fat cells, which are enclosed membranes filled with globules of triglycerides. In normal fat the fat cells have regular contours and form into grapelike clusters. The intracellular fat is relatively fluid and, if the membrane is pierced, will flow out of the cell into the interstitial space. The interstitial space includes nerves, blood vessels, lymphatics and collagen fibers, among other substances. Liposuction is performed by inserting a narrow tube, or cannula, through a tiny incision in the skin into the subcutaneous fatty tissue. The cannula is repeatedly pushed then pulled through the fat layer, separating and puncturing the fat cells and suctioning them out. Suction action through the cannula is provided by a vacuum pump or a large syringe. The procedure carries with it some risks and side effects. Due to the physical damage induced, the procedure can damage nerves, lymphatics and vasculature in the surrounding area, often resulting in significant loss of blood as the blood is vacuumed out with the fat and the formation of seroma due to damaged lymphatic channels. In addition, the post-procedure recovery period is long and often accompanied by a great deal of inflammation, bruising and concomitant pain.

Since the liposuction technique was first developed there have been many improvements to the technique, with the goal of making the surgery less dangerous for the patient, as well as reducing the negative aspects of the post-operative recovery period. For example, in the tumescent technique known in prior art, a saline solution containing very dilute amounts of at least an anesthetic and a vasoconstrictor is injected subcutaneously into the area to be suctioned. The anesthetic reduces operative and post-operative pain and the vasoconstrictor helps reduce blood loss. Cannulas have been improved by enabling the cannula to emit laser light and ultrasound energy directly onto the fat cells. This internal application of energy melts the cell wall, releasing the intracellular fat, thereby making the fatty tissue less viscous and more easily suctioned up through the narrow cannula. These procedures suffer the disadvantage of still having to physically stab the cannula repeatedly in the fat layer as well as essentially melting the adipose tissue, resulting in undesirable levels of bruising, inflammation, pain, blood loss, and seroma formation. Recovery time is significant.

In U.S. Pat. No. 6,605,079, issued to one of the inventors of this method and incorporated herein, a less-destructive method is disclosed that uses low energy laser therapy in conjunction with suction of the fat cells. Low level laser therapy (LLLT) has been used increasingly in the treatment of a broad range of conditions such as treatment and repair of injured muscles and tendons. LLLT has improved wound healing, reduced edema, and relieved pain of various etiologies. LLLT has been used successfully post-operative to liposuction to reduce inflammation and pain. While a significant improvement over prior art, it is still invasive and carries with it the corresponding pain and risks.

Non-invasive methods of fat reduction are preferred over invasive methods to minimize trauma to the patient, reduce the risk of infection, and speed up recovery time, among other reasons. To that end, topical agents have long been known which claim to reduce cellulite or at least the appearance of cellulite. The effect of these agents on cellulite is somewhat dubious, and these agents are not known to actually reduce fat. Some of the topical agents are used in combination with massage or radiation of the affected areas.

To avoid invasive procedures, electromagnetic energy, such as microwave, ultrasound or radio frequency radiation, has also been used to reduce fat. In U.S. Pat. No. 5,507,790 issued to Weiss, a method is described in which a medicament is applied to a patient's skin where fat removal is desired and focused electromagnetic energy is applied to the same work site to heat the fatty tissue and increase fat lipolysis. In U.S. Pat. No. 5,143,063, Fellner takes this method even farther, applying sufficient electromagnetic radiation to destroy the fat cells. Yet another method is to inject an intumescing solution below the skin and apply electromagnetic energy externally to the body. These procedures are disadvantageous in that they utilize such high energy sources that they excessively heat the surrounding tissue, which can result in damage to the tissue and pain. Again, recovery time is significant.

Other external applications of certain types of destructive energy are known in the art. U.S. Pat. No. 6,645,162 issued to Friedman, et al. discloses the superposition of ultrasound waves from two or more sources to create a wave having high intensity localized at the adipose tissue to be treated. With this method, fat cells are sonically disintegrated, allowing the body to dispose of the fat that has been freed. In addition to destruction of cells, another difficulty with this method is accurately obtaining the desired focal zone under the skin.

Co-pending patent application Ser. No. 11/053,369 discloses the use of low-level laser energy applied externally to the patient to release at least a portion of the intracellular fat into the interstitial space, wherein the released fat and damaged fat cells are removed from the patient's body through one or more of the patient's normal bodily systems. While effective, it would be desirable to prevent the fat cells from recuperating before they are removed from the body. To that purpose, the cells must be completely destroyed or recuperation delayed for sufficient time to be removed from the body, so that the cells do not repair and refill with fat.

It is desirable to remove fat with less damage to the fatty tissue, less blood loss, less post-operative bruising, inflammation, and pain than existing methods. It is desirable to eliminate fat cells so that they do not repair and refill with fat before they are removed from the body. Therefore, an object of this invention is to provide a non-invasive method of reducing fat. Another object is to provide a non-invasive method of reducing fat that does not damage surrounding tissue or structures. Another object is to provide a non-invasive method of reducing fat that helps eliminate fat cells before they recuperate. It is another object to eliminate the need for recovery time.

### SUMMARY OF THE INVENTION

This invention is a noninvasive method of reducing fat from targeted regions of a patient's body by applying low-level laser energy externally through the skin of the patient to the targeted areas and treating the patient with a catalyst to prevent the lasered fat cells from functioning normally. Sufficient laser energy is applied to release at least a portion of intracellular fat into the interstitial space and the preferred embodiment uses laser light at about 635 nm. The catalyst is any combination of sterile water and saline solution that kills the lasered fat cells or prevents recuperation of the lasered cells before they are removed from the body through the body's natural functions. Preferably the catalyst is a hypotonic saline solution.

### BRIEF DESCRIPTION OF THE DRAWINGS

FIG. 1 is illustrates the application of low-level laser radiation and catalyst.

FIG. 2 is a schematic illustration of normal fat cells.

FIG. 3 is a schematic illustration of fat cells after externally-applied low-level laser radiation.

FIG. 4 is a top-view of a multi-laser scanner.

### DETAILED DESCRIPTION OF THE INVENTION

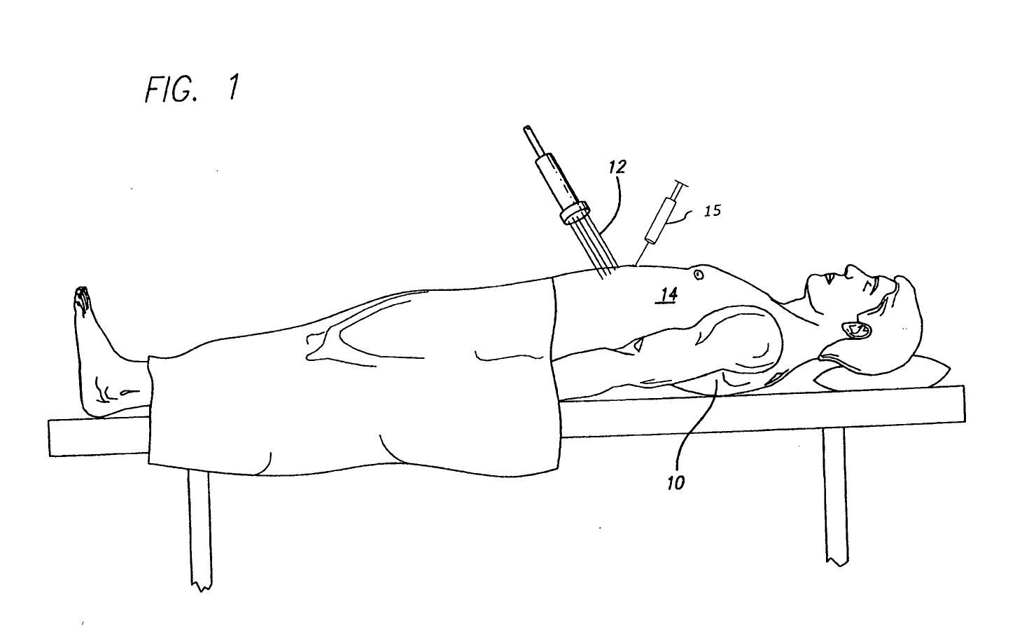

This invention is a method for removing adipose tissue from a patient's body 10. As illustrated in FIG. 1, laser energy 12 is applied to the adipocyte tissue externally through the skin 14 of the patient. Sufficient laser energy is applied to release at least a portion of the intracellular fat 23 into the interstitial space 32. Further, the lasered cells are treated with a substance that kills the lasered fat cells or prevents recuperation of the lasered cells before they are removed from the body through the body's natural functions. This substance is referred to herein as a catalyst. The released intracellular fat and damaged fat cells are removed from the body through the body's normal systems, such as metabolic, lymphatic or excretory systems. The procedure may be repeated in one or more additional areas to remove additional fat there.

Typically, fat leakage into the interstitial space is seen as early as 3-5 minutes of laser energy application. This leakage continues for treatments as long as about 12-15 minutes with no fat cell destruction. However, at treatments of over about 12 minutes, fat cells start being destroyed. Therefore, when destruction of fat cells is not desired, the preferred method of treatment is to apply repeated treatments up to 12 minutes each and more preferably at 12 minutes each. Conversely, for patients with more fat to treat, it may be desirable to destroy the fat cells so that it cannot recover and reaccumulate fat. In such case, sufficient laser energy is applied to destroy fat cells without heating them or surrounding tissue. That is, for destructive treatments, each treatment will be at least 12 minutes and preferably 15-20 minutes.

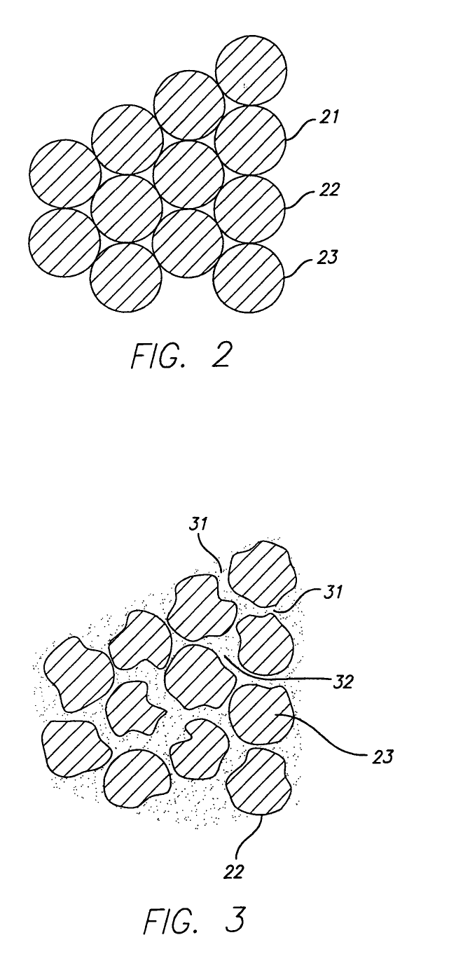

The mechanism involved in releasing the intracellular fat from the cells is believed to be the formation of a transitory pore in the cell membrane. FIG. 2 illustrates adipose tissue comprising normal fat cells 21 wherein the cell membrane 22 is filled with intracellular fat 23. Upon sufficient doses of low-level laser energy, the cell membrane 22 is momentarily disrupted, releasing the intracellular fat 23. See FIG. 3, which illustrates pores 31 in the cellular membrane 22 which have released intracellular fat 23 into the interstitial space 32. Upon cessation of the energy application, the pores 31 close and the cell membrane 22 returns to contiguity. The fat cell is not destroyed, provided the duration of laser treatment is appropriate. For a 635 nm laser of less than 1 W, treatments of less than about 12 minutes do not destroy cells.

The biochemical effect of applying the low-level laser energy has been proven to stimulate the mitochondria of the adipocyte cells. Following the stimulation of the mitochondria, there is an increase in the production of ATP. The newly synthesized ATP triggers the up-regulation of cyclic adenosine monophosphate (cAMP). cAMP has been shown to stimulate cytoplasmic lipase, triggering the conversion of triglycerides into fatty acids and glycerol that can easily pass through the cell membrane. The transitory pore is evidence that the laser is allowing for the movement of fatty acids, glycerol, and triglycerides to pass across the membrane and into interstitial space. Additionally, it is believed applying the low-level laser energy causes the vasodilation of nearby blood vessels and arteries, allowing for the absorption of the newly released fatty acids and triglycerides.

Before, during or after the laser treatment, the patient is also treated with a catalyst. The catalyst can be administered in any manner that enables it to reach the fat cells, for example transdermally by injection or skin absorption, orally, nasally, or intravenously. In the preferred embodiment, the catalyst is injected at and around the area targeted for fat reduction. FIG. 1 shows a catalyst being injected with a syringe 15. The catalyst can be administered with one injection per treatment area or multiple injections per treatment area. In the preferred embodiment, a total of 5 cc of the catalyst is administered to the patient via seven injections for the treatment area: 6 injections of 0.8 cc and 1 injection of 0.2 cc. Injections are given with a ½ inch 27 gauge needle at the subcutaneous fat level (usually at 1 cm) and are spaced approximately 2 cm apart to span an area of approximately 80 cm².

One form of the catalyst is a hypotonic solution. A hypotonic solution is a solution that contains less dissolved salt than that of the cellular content. The administration of hypotonic solution after lasering acts to further propel the movement of fatty acids, glycerol, and triglycerides across the cell membrane and into the interstitial space. Because cell membranes are permeable to water, when a cell is placed in a hypotonic solution, the water diffuses into the cell through osmosis. If the difference in concentration is significantly high, the cell walls rupture with an influx of too much water, leading to the death of the cell. The fat cell “drains away” harmlessly via the lymphatic system. Surrounding critical structures such as skin, blood vessels, nerves and connective tissue remain intact. In the preferred embodiment, the hypotonic solution consists of 70% saline and 30% water. In another embodiment, the catalyst is a hypotonic saline solution of 0.9% w/v NaCl or less. In a second embodiment, the catalyst is a buffered saline solution, such as phosphate buffered saline (NaCl, a sodium phosphate, and a potassium phosphate). Alternatively, the hypotonic solution is simply sterile water, which has no electrolyte concentration and therefore maximizes the influx of water into the cell.

Another form of the catalyst is a hypertonic solution, which contains a higher concentration of electrolytes than that found in fat cells. If such a solution is allowed to enter the blood stream, the osmotic pressure difference between the blood and the cells will cause water to flow out of the fat cells, which will then shrink. This causes serious damage to the cells that takes a relatively long time to repair, and it may also lead to the death of the cell. Another embodiment of the catalyst is a hypertonic saline solution of more than 0.9% w/v NaCl. In yet another embodiment, the catalyst is a hypertonic buffered solution, such as phosphate buffered saline (NaCl, a sodium phosphate, and a potassium phosphate).

The laser energy applied is low level, that is, the treatment has a dose rate that causes no immediate detectable temperature rise of the treated tissue and no macroscopically visible changes in tissue structure. The laser energy penetrates the skin and is specific to the depth of the desired zone of fat to be treated. Consequently, the treated and surrounding tissue is not heated and is not damaged. Preferably the laser light is visible to the human eye so that the area of application is easily determined. A laser device that provides this low-level energy is known in the art as a cold laser, such as the inventions described in U.S. Pat. Nos. 6,013,096 issued to Tucek and 6,746,473, issued to Tucek and Shanks. Other lasers known in the art for use in low-level laser therapy include Helium-Neon lasers having a 632 nm wavelength and semiconductor diode lasers with a broad range of wavelengths between 405-1500 nm. The laser device may have one or more laser energy sources. Different therapy regimens require diodes of different wattages. The preferred laser diodes use less than one watt of power each to simultaneously facilitate liposuction, treat post-operative inflammation, and post-operative pain. Diodes of various other wattages may also be employed to achieve the desired laser energy for the given regimen. Low-level lasers are available commercially.

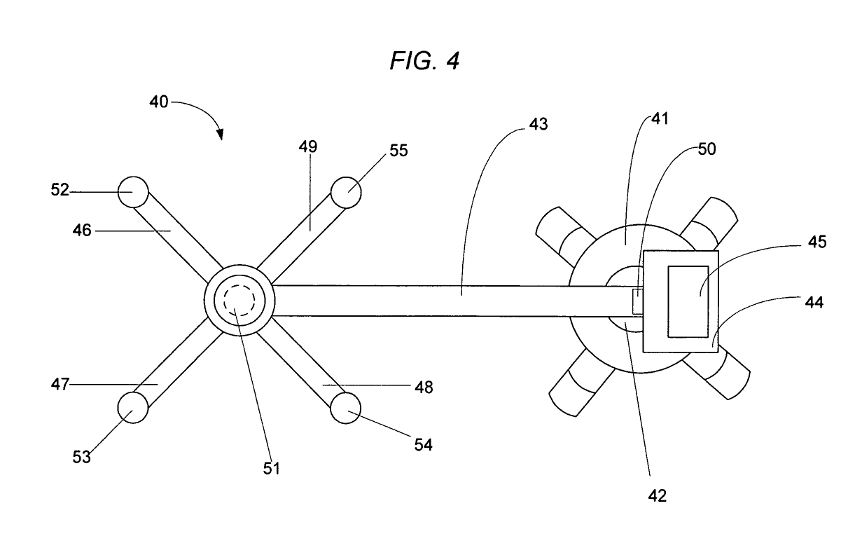

Another laser device particularly useful for this application is the stand-alone multi-laser scanner described in US Patent Publication 2006/0095099 belonging to Shanks and Tucek. FIG. 4 illustrates an embodiment of the multi-laser scanner for use with this laser application. Additional embodiments of a multi-laser scanner can be used as well. As shown in FIG. 4, the multi-laser scanner 40 is supported by a wheeled base 41. Preferably the wheeled base includes at least two locking wheels to ensure affixed placement. Additionally, the wheeled base preferably houses a charger jack for the unit and the mechanical components required to operate the multi-laser scanner. A height-adjustable vertical support 42 extends upwards from wheeled base 41. A freely rotatable boom arm 43 extends outward from vertical support 42. Preferably boom arm 43 is a two-jointed arm that offers placement flexibility with a bend off the base and before the head. Descending from the boom arm is the laser scanner unit which comprises four scanner arms 46, 47, 48, and 49. The scanner arms protrude from the center of the laser scanner unit and are flexible arms that allow for 90 degree rotation. At the center of the laser scanner unit is a first laser head 51. Attached to each scanner arm are additional laser heads 52, 53, 54 and 55. Each laser head contains an independent diode of variable frequency and preferably having a 635 nm wavelength. The four heads 52, 53, 54, and 55 attached to scanner arms 46, 47, 48, and 49 are positioned 90 degrees apart from each other, and each is tilted at a 30 degree angle from the centerline of the center scanner. Each of the independent laser diodes is processed through a lens that redirects the beam with a line refractor. The refracted light is then bent into a spiraling circle pattern that is random and independent of the other diodes. These patterns overlap each other to provide coverage within the target area. Each laser head emits 17 mW, 635 nm of red laser light. Multi-laser scanner 40 also includes, in the preferred embodiment, a control center 44 and touch screen 45 that acts as the command module and the user's interface with the device. Additionally, a key lock 50 is positioned on multi-laser scanner 40 to lock the device. The unit is turned on with key lock 50. Additional technical details of the multi-laser scanner are disclosed in US Patent Publication 2006/0095099.

The dosage of laser energy required to achieve release of the intracellular fat into the interstitial space will vary depending on the thickness of the patient's skin, thickness of fatty tissue, and other biological factors peculiar to each patient. The following examples are illustrative:

### EXAMPLE 1

The targeted area for fat reduction is the patient's abdomen. The patient is treated with injections totaling 5 cc of sterile water. The sterile water is administered to the patient via seven injections for the treatment area: 6 injections of 0.8 cc and 1 injection of 0.2 cc. Injections are given with a ½ inch 27 gauge needle at a depth of about 1 cm and are spaced approximately 2 cm apart to span an area of approximately 80 cm². The patient's abdomen is then treated with laser energy, using a 635 nm semiconductor diode laser with maximum power of 10 mW. The laser energy is applied for 12 minutes in a back-and-forth sweeping motion across the targeted fat areas without touching the patient.

### EXAMPLE 2

The targeted area for fat reduction is the patient's abdomen. The patient is treated with a 0.9% w/v NaCl solution. The patient is treated with injections totaling 5 cc of the solution. The solution is administered to the patient via seven injections for the treatment area: 6 injections of 0.8 cc and 1 injection of 0.2 cc. Injections are given with a ½ inch 27 gauge needle at a depth of about 1 cm and are spaced approximately 2 cm apart to span an area of approximately 80 cm². The patient's abdomen is then treated with laser energy, using a 635 nm semiconductor diode laser with maximum power of 10 mW. The laser energy is applied for 12 minutes in a back-and-forth sweeping motion across the targeted fat areas without touching the patient.

### EXAMPLE 3

The targeted area for fat reduction is the patient's abdomen. A 635 nm semiconductor diode laser with maximum power of 10 to 20 mW is used to apply laser light to the targeted area. The laser energy is applied for 12-15 minutes in a back-and-forth sweeping motion across the targeted fat area without touching the patient. The patient is then treated with injections totaling 5 cc of sterile water. The solution is administered to the patient via seven injections for the treatment area: 6 injections of 0.8 cc and 1 injection of 0.2 cc. Injections are given with a ½ inch 27 gauge needle at a depth of about 1 cm and are spaced approximately 2 cm apart to span an area of approximately 80 cm². This method is repeated four times, spaced about a week apart, over a four week period.

### EXAMPLE 4

The targeted area for fat reduction is the patient's abdomen. A 635 nm semiconductor diode laser with maximum power of 10 mW is used to apply laser light to the targeted area. The laser energy is applied for 12-15 minutes in a back-and-forth sweeping motion across the targeted fat area without touching the patient. The patient is treated with a hypotonic solution consisting of 70% saline and 30% water. The patient is treated with injections totaling 5 cc of the solution. The solution is administered to the patient via seven injections for the treatment area: 6 injections of 0.8 cc and 1 injection of 0.2 cc. Injections are given with a ½ inch 27 gauge needle at a depth of about 1 cm and are spaced approximately 2 cm apart to span an area of approximately 80 cm². This method is repeated four times, spaced about a week apart, over a four week period.

### EXAMPLE 5

The targeted area for fat reduction is the patient's abdomen. The center diode of the multi-laser scanner is positioned at a distance of 6.00 inches above the patient's abdomen, centered along the body's midline (the “line” that vertically “dissects” the body into two equal halves) and focused on the navel. The other four diodes are positioned 90 degrees apart and tilted 30 degrees off the centerline of the center scanner. The multi-laser scanner is activated for 12 minutes and each scanner emits to the subject a laser beam of approximately 5 mW with a wavelength of 635 nm. Each laser beam is applied in a spiraling circle pattern that is totally random and independent from the others. The patterns overlap to guarantee total coverage within the target area of approximately 8 by 10 inches. The patient is treated with a hypotonic solution consisting of 70% saline and 30% water. The patient is treated with injections totaling 5 cc of the solution. The solution is administered to the patient via seven injections for the treatment area: 6 injections of 0.8 cc and 1 injection of 0.2 cc. Injections are given with a ½ inch 27 gauge needle at a depth of about 1 cm and are spaced approximately 2 cm apart to span an area of approximately 80 cm². This method is repeated four times, spaced about a week apart, over a four week period.

### EXAMPLE 6

The targeted area for fat reduction is the patient's hips. The center diode of the multi-laser scanner is positioned at a distance of 6.00 inches above the center of the first treatment region, the patient's right hip area. The other four diodes are positioned 90 degrees apart and tilted 30 degrees off the centerline of the center scanner. The multi-laser scanner is activated for 12 minutes and each scanner emits to the subject a laser beam of approximately 5 mW with a wavelength of 635 nm. Each laser beam is applied in a spiraling circle pattern that is totally random and independent from the others. The patterns overlap to guarantee total coverage within the target area of approximately 8 by 10 inches. The patient is treated with a hypotonic solution consisting of 70% saline and 30% water. The patient is treated with injections totaling 5 cc of the solution. The solution is administered to the patient via seven injections for the treatment area: 6 injections of 0.8 cc and 1 injection of 0.2 cc. Injections are given with a ½ inch 27 gauge needle at a depth of about 1 cm and are spaced approximately 2 cm apart to span an area of approximately 80 cm². The procedure is then repeated for the second treatment region, the patient's left hip area. This method is repeated four times, spaced about a week apart, over a four week period.

### EXAMPLE 7

The targeted area for fat reduction is the patient's outer thighs. The center diode of the multi-laser scanner is positioned at a distance of 6.00 inches above the center of the first treatment region, the outer thigh on the right side of the patent. The other four diodes are positioned 90 degrees apart and tilted 30 degrees off the centerline of the center scanner. The multi-laser scanner is activated for 12 minutes and each scanner emits to the subject a laser beam of approximately 5 mW with a wavelength of 635 nm. Each laser beam is applied in a spiraling circle pattern that is totally random and independent from the others. The patterns overlap to guarantee total coverage within the target area of approximately 8 by 10 inches. The patient is treated with a hypotonic solution consisting of 70% saline and 30% water. The patient is treated with injections totaling 5 cc of the solution. The solution is administered to the patient via seven injections for the treatment area: 6 injections of 0.8 cc and 1 injection of 0.2 cc. Injections are given with a ½ inch 27 gauge needle at a depth of about 1 cm and are spaced approximately 2 cm apart to span an area of approximately 80 cm². The procedure is then repeated for the second treatment region, the outer thigh on the left side of the patient. This method is repeated four times, spaced about a week apart, over a four week period.

While there has been illustrated and described what is at present considered to be a preferred embodiment of the present invention, it will be understood by those skilled in the art that various changes and modifications may be made, and equivalents may be substituted for elements thereof without departing from the true scope of the invention. Therefore, it is intended that this invention not be limited to the particular embodiment disclosed as the best mode contemplated for carrying out the invention, but that the invention will include all embodiments falling within the scope of the appended claims.

We claim:

1. A method for reducing fat of a patient in a targeted area, the method comprising:
a) applying laser energy externally to the patient at the targeted area; and
b) treating the fat cells in the targeted area with a catalyst.

2. The method of claim 1 wherein the catalyst is composed of only saline.

3. The method of claim 1 wherein the catalyst is a hypotonic saline solution.

4. The method of claim 2 wherein the catalyst comprises less than or equal to 0.9% w/v NaCl.

5. The method of claim 2 wherein the catalyst comprises more than 0.9% w/v NaCl.

6. The method of claim 1 wherein the catalyst is a hypertonic saline solution.

7. The method of claim 1 wherein the catalyst is composed of only sterile water.

8. The method of claim 1 wherein the catalyst destroys the fat cells.

9. The method of claim 1 wherein the laser energy is applied to the skin of the patient at and around the site where fat is to be reduced.

10. The method of claim 1 wherein the laser energy is generated by a semiconductor diode.

11. The method of claim 1 wherein the laser energy is in the visible spectrum.

12. The method of claim 1 wherein the laser energy is about 635 nm.

13. The method of claim 1 wherein the laser energy is provided by a laser device having power of less than 1 watt.

14. The method of claim 1 in which the application of laser energy occurs before the treatment with the catalyst.

15. The method of claim 1 in which the application of laser energy occurs after the treatment with the catalyst.

16. A method for reducing fat of a patient in a targeted area, wherein the fat comprises fat cells having intracellular fat and interstitial space between the fat cells, the method comprising:
a) using a laser emitting light at about 635 nm to apply one or more treatments totaling less than 12 minutes of laser energy externally to the patient to release at least a portion of the intracellular fat into the interstitial space; and
b) treating the fat cells in the targeted area with a catalyst;
wherein the released fat and damaged fat cells are removed from the patient's body through one or more of the patient's normal bodily systems.

17. The method of claim 16 in which the application of laser energy occurs before the treatment with the catalyst.

18. The method of claim 16 in which the application of laser energy occurs after the treatment with the catalyst.

19. The method of claim 16 wherein the catalyst is a hypotonic solution composed of only saline.

20. The method of claim 16 wherein the catalyst is a hypertonic solution composed of only saline.

21. The method of claim 16 wherein the catalyst is sterile water.

22. The method of claim 16 wherein the laser is hand-held.

23. The method of claim 16 wherein the laser is a stand-alone laser scanner.
